(19) United States
(12) Patent Application Publication
(10) Pub. No.: US 2026/0177556 A1
(43) Pub. Date: Jun. 25, 2026

(54) PROXIMITY-BASED LABELING OF SIALYLATED GLYCOPROTEINS

(71) Applicant: THE TRUSTEES OF PRINCETON UNIVERSITY, Princeton, NJ (US)

(72) Inventors: Steven Douglas Knutson, Princeton, NJ (US); Ciaran Paul Seath, Palm Beach Gardens, FL (US); Claudio Flavio Meyer, Basel (CH); David W.C. MacMillan, Princeton, NJ (US)

(21) Appl. No.: 19/124,667

(22) PCT Filed: Oct. 26, 2023
(86) PCT No.: PCT/US2023/077910

§ 371 Date: Apr. 25, 2025

Related U.S. Application Data
(60) Provisional application No. 63/419,519, filed on Oct. 26, 2022.

(51) Int. Cl. G01N 33/68; G01N 33/543; G01N 33/58

(52) U.S. Cl. CPC G01N 33/6803; G01N 33/54306; G01N 33/58; G01N 33/6842

(57) ABSTRACT

Systems and methods are described herein enabling the profiling of local microenvironments across the sialylated proteome via proximity labeling. In one aspect, conjugates are described herein having composition and electronic structure for generating reactive labeling intermediates in microenvironments of sialylated cell-surface glycoproteins. In some embodiments, a conjugate comprises a transition metal catalyst coupled to a cell surface glycoprotein. As described further herein, the transition metal catalyst can be coupled to the glycoprotein via a derivatized sialic acid linker.

### RELATED APPLICATION DATA

The present application claims priority pursuant to Article 8 of the Patent Cooperation Treaty to U.S. Provisional Patent Application Ser. No. 63/419,519 filed Oct. 26, 2022 which is incorporated herein by reference in its entirety.

### FIELD

The present invention relates to compositions, systems, and methods for proximity-based labeling and, in particular, for proximity-based labeling to profile local microenvironments across the sialylated proteome.

### BACKGROUND

Glycosylation is one of the most common post translational modifications (PTM) on proteins, occurring on at least 50% of all known mammalian proteins and dramatically increasing the functional proteome. Glycosylation can alter both protein localization and function, and misregulated deposition has been shown to contribute to varied disease phenotypes, such as cancer metastasis, viral immune escape, viral entry, and inflammation. Glycoproteins also play a critical role in overall cell-surface architecture, contributing to cell adhesion, cell signaling, viral docking and cell-cell interactions. Among the array of cell-surface monosaccharides, sialic acid stands out as being particularly influential for cell function. This charged sugar is incorporated by sialyltransferases, and commonly decorates the termini of polysaccharide chains. During oncogenesis, overexpression of sialyltransferases leads to hypersialylation, in turn promoting tumor progression through two different paradigms: (1) Sialylation appears to inhibit apoptosis and allow the cell to evade the immune system, and (2) The sialoglycoconjugate sialyl Lewisx facilitates metastasis via extravasation of cancer cells out of the bloodstream into nearby tissue.

Despite these observations, the underlying biochemical mechanisms remain poorly understood, in part due to a lack of high-resolution tools to assess the functional roles of sialylation.

### SUMMARY

In view of the foregoing disadvantages, systems and methods are described herein enabling the profiling of local microenvironments across the sialylated proteome via proximity labeling. In one aspect, conjugates are described herein having composition and electronic structure for generating reactive labeling intermediates in microenvironments of sialylated cell-surface glycoproteins. In some embodiments, a conjugate comprises a transition metal catalyst coupled to a cell surface glycoprotein. As described further herein, the transition metal catalyst can be coupled to the glycoprotein via a derivatized sialic acid linker. In some embodiments, the transition metal catalyst and derivatized sialic acid linker are coupled via click chemistry. Suitable click chemistry moieties of the transition metal complex and/or derivatized sialic acid linker can be selected from the group consisting of DBCO, BCN, TCO, tetrazine, alkyne and azide. For example, sialic acid can be derivatized to include a suitable click chemistry moiety for coupling with the transition metal catalyst. FIG. 1 illustrates sialic acid derivatized with an azide functionality for reaction with the DBCO moiety of the transition metal catalyst.

In some embodiments, the derivatized sialic acid linker is metabolically incorporated to cell surface glycoproteins, followed by reaction to capture the transition metal catalyst. FIG. 1 illustrates a non-limiting embodiment wherein azide derivatized sialic acid linker is metabolically incorporated to cell surface glycoproteins. The transition metal catalyst can comprise a platinum group metal center, in some embodiments. Moreover, in some embodiments, the transition metal catalyst is of Formula I:

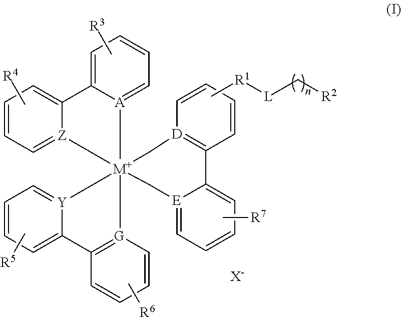

wherein M is a transition metal,
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and R⁹OH, wherein R⁸ is selected from the group consisting of hydrogen and alkyl, and R⁹ is alkyl; wherein Rt is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20. As provided in Formula I, the linking moiety, L, is optional and, therefore, may not be present in some embodiments of the transition metal catalyst.

Polarity of the transition metal complexes can be tailored to specific cellular environments via selection of R³-R⁷. In some embodiments, for example, one or more of R³-R⁷ are selected to exhibit hydrophilic character via charged and/or polar chemical moieties. In such embodiments, the transition metal complex can exhibit hydrophilic character suitable for placement in intercellular/extracellular environments. Transition metal complexes illustrated in FIG. 6, for example, incorporate charged and polar chemical moieties for the aqueous intercellular environment. Alternatively, the one or more of R³-R⁷ are selected to exhibit hydrophobic, lipophilic, or non-polar character.

The transition metal catalyst can have electronic structure for energy transfer to a protein labeling agent to produce a reactive intermediate. In some embodiments, the energy transfer is Dexter energy transfer or electron transfer. Energy transfer to the protein labeling agent can originate from an excited state of the transition metal catalyst electronic structure, in some embodiments. The excited state of the catalyst, for example, can be a singlet excited state or triplet excited state. The excited state of the catalyst can be generated by one or more mechanisms, including energy absorption by the catalyst. In some embodiments, the catalyst is a photocatalyst, wherein the excited state is induced by absorption of one or more photons. In other embodiments, the catalyst may be placed in an excited state by interaction with one or more chemical species in the surrounding environment. Alternatively, energy transfer to the protein labeling agent, including electron transfer, may originate from a ground state of the catalyst electronic structure.

In another aspect, systems for profiling microenvironments local to the sialylated proteome are provided. A system, in some embodiments, comprises a protein labeling agent, and a conjugate including a transition metal catalyst coupled to a cell surface glycoprotein via a derivatized sialic acid linker, wherein the transition metal catalyst has electronic structure permitting energy transfer to the protein labeling agent to provide a reactive intermediate. The reactive intermediate is operable to label a protein or other biomolecule within a predetermined radius of the conjugate. The predetermined radius may be a diffusion radius of the reactive intermediate.

The diffusion radius of the reactive intermediate can be tailored to specific microenvironment mapping (proximity-based labeling) considerations, and can be limited to the nanometer scale. In some embodiments, for example, the diffusion radius of the reactive intermediate can be less than 100 nm, less than 50 nm, less than 10 nm, less than 5 nm, less than 4 nm, less than 3 nm, or less than 2 nm prior to quenching in the surrounding environment. The diffusion radius can be 0.5 nm to 10 nm, in some embodiments. Accordingly, the reactive intermediate will react or crosslink with a protein or other biomolecule within the diffusion radius or be quenched by the surrounding environment if no protein or biomolecule is present. In this way, high resolution of the local environment can be mapped via concerted effort between the catalyst and protein labeling agent. Additionally, the reactive intermediate can exhibit a t_{1/2} less than 5 ns, less than 4 ns, or less than 2 ns prior to quenching, in some embodiments. The reactive intermediate, for example, can exhibit a t_{1/2} less of 1-5 ns. In additional embodiments, the diffusion radius can be extended to between 5-500 nm though extension of the reactive intermediate half-life. For example, in some embodiments, the reactive intermediate can have a half-life of 1-100 μs, or greater.

In some embodiments, the protein labeling agent can be a diazirine. Triplet energy transfer from the excited state photocatalyst can promote the diazirine to its triplet (T₁) state. The diazirine triplet under-goes elimination of N₂ to release a free triplet carbene, which undergoes picosecond-timescale spin equilibration to its reactive singlet state (t_{1/2}<1 ns) which either crosslinks with a nearby protein or is quenched in the aqueous environment. In some embodiments, the extinction coefficient of the transition metal complex is 3 to 5 orders of magnitude greater than that of the diazirine.

Any diazirine consistent with the technical principles discussed herein. Diazirine sensitization, for example, can be extended to a variety of p- and m-substituted aryltrifluoromethyl diazirines bearing valuable payloads for microscopy and proteomics applications, including free carboxylic acid, phenol, amine, alkyne, carbohydrate, and biotin groups. The diazirine can be functionalized with a marker, such as biotin. In some embodiments, the marker is desthiobiotin. The marker can assist in identification of proteins labeled by the protein labeling agent. The marker, for example, can be useful in assay results via western blot and/or other analytical techniques. Markers can include alkyne, azide, FLAG tag, fluorophore, and chloroalkane functionalities, in addition to biotin and desthiobiotin.

In additional embodiments wherein the transition metal catalyst is a photocatalyst, the protein labeling agent can be an azide. Triplet energy transfer from the excited state photocatalyst can promote nitrene formation from the azide. The reactive nitrene either crosslinks with a nearby protein or is quenched in the aqueous environment. Any azide operable to undergo energy transfer with eth transition metal photocatalyst for nitrene formation can be employed. In some embodiments, an azide is an aryl azide.

In another aspect, methods of profiling microenvironments local to the sialylated proteome are provided. A method comprises forming a conjugate comprising a transition metal catalyst coupled to a cell surface glycoprotein via a derivatized sialic acid linker, and activating a protein labeling agent to a reactive intermediate with the transition metal catalyst. The reactive intermediate is coupled to a protein or other biomolecule within a predetermined radius of the conjugate. The transition metal catalyst, protein labeling agent, and reactive intermediate can have any composition and/or properties described herein.

These and other embodiments are further described in the following detailed description.

### BRIEF DESCRIPTION OF THE DRAWINGS

FIG. 1 illustrates using microenvironment mapping with a system described herein comprising a conjugate including a transition metal catalyst coupled to a cell surface glycoprotein via a derivatized sialic acid linker, and a protein labeling agent, wherein the transition metal catalyst has electronic structure permitting energy transfer to the protein labeling agent to provide a reactive intermediate.

FIG. 2A illustrates workflow for the glycomap experiments. Ac4ManNAz incubation performed for 72 h, however shorter times led to equivalent results in the cell lines in this study. Optimization of Ir-DBCO incubation time is shown in the supporting information (FIG. S3) of the Appendix.

FIG. 2B provides Western blot analysis of whole cell lysates after glycomap experiment.

FIG. 2C provides immunofluorescence analysis of cells after glycomap experiment; red: streptavidin, blue: hoechst.

FIG. 2D is Western blot of streptavidin enriched lysate, stained against Nicastrin (top lane) and CD55 (bottom lane).

FIG. 3A illustrates workflow for TMT-based chemoproteomic discovery of interactome of sialylated glycoproteins. Each experiment was performed in triplicate.

FIG. 3B provides quantitative chemoproteomics validation by glycomap of HEK293T cells. For all experiments the same cutoffs (>1.5 Log₂(fold change); >1.5−Log₁₀(p-value)) were used.

FIG. 4A summarizes comparative proteomics experiment of primary cervical cells (PCC) and HeLa cells. Top row: sialylated glycoproteins in PCC (left) and HeLa (right). Bottom row: interacting proteins in PCC (left) and HeLa cells (right). Middle: Venn diagram of the enriched proteins from each dataset. The same cutoffs (>1.5 Log₂(fold change); >1.5−Log₁₀(p-value)) were used for the analysis of all data sets.

FIG. 4B is gene ontology (GO) analysis of the identified sialylated glycoproteins (top) and their interactors (bottom).

FIG. 4C is a Venn diagram of the enriched solute carrier proteins (SLCs) that interact with sialylated glycoproteins.

FIG. 5A illustrates workflow for the metabolomics analysis of HeLa cells.

FIG. 5B quantifies Metabolite levels of selected small molecules. Experiments were performed in triplicate.

FIG. 5C—Left: GO analysis suggests cation homeostasis is affected by sialylation. Middle: enriched zinc transporters in the HeLa and PCC dataset. Right: Colorimetric zinc assay, which shows a significant change in cellular zinc levels in response to desialylation. P-Values determined by unpaired students t-test. *P<0.05, **P<0.01.

FIG. 6 illustrates transition metal complexes incorporating charged and/or polar chemical moieties for the aqueous intercellular environment, according to some embodiments.

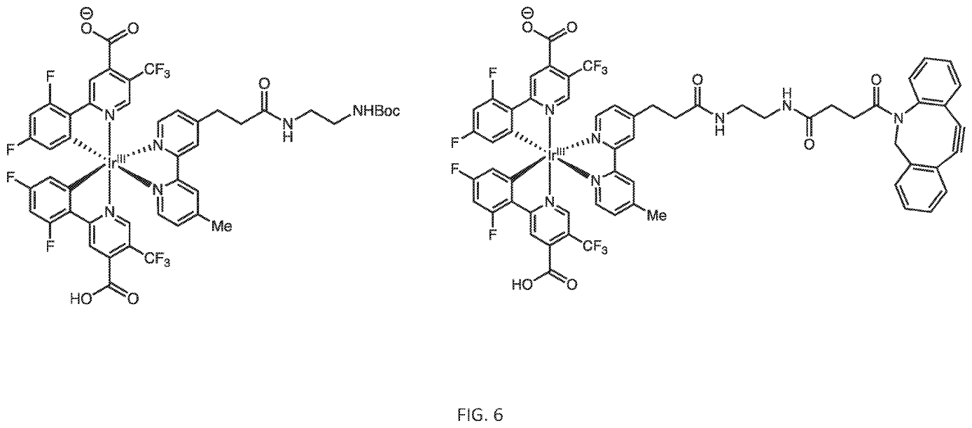

FIG. 7 illustrates various chemical species employed in some embodiments of compositions and methods described herein.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “alkynyl” as used herein, alone or in combination, refers to a straight or branched chain hydrocarbon group having at least one carbon-carbon triple bond and optionally substituted with one or more substituents.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, boron, oxygen and/or sulfur.

The term “heterocycle” as used herein, alone or in combination, refers to a mono- or multicyclic ring system in which one or more atoms of the ring system is an element other than carbon, such as boron, nitrogen, oxygen, and/or sulfur or phosphorus and wherein the ring system is optionally substituted with one or more ring substituents. The heterocyclic ring system may include aromatic and/or non-aromatic rings, including rings with one or more points of unsaturation.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as boron, nitrogen, oxygen, sulfur or phosphorus, alone or in combination, and wherein the ring system is optionally substituted with one or more ring substituents.

The term “alkoxy” as used herein, alone or in combination, refers to the moiety RO—, where R is alkyl, alkenyl, or aryl defined above.

The term “halo” as used herein, alone or in combination, refers to elements of Group VIIA or Group 17 of the Periodic Table (halogens). Depending on chemical environment, halo can be in a neutral or anionic state.

Terms not specifically defined herein are given their normal meaning in the art.

Embodiments of the present application are further illustrated in the following non-limiting examples.

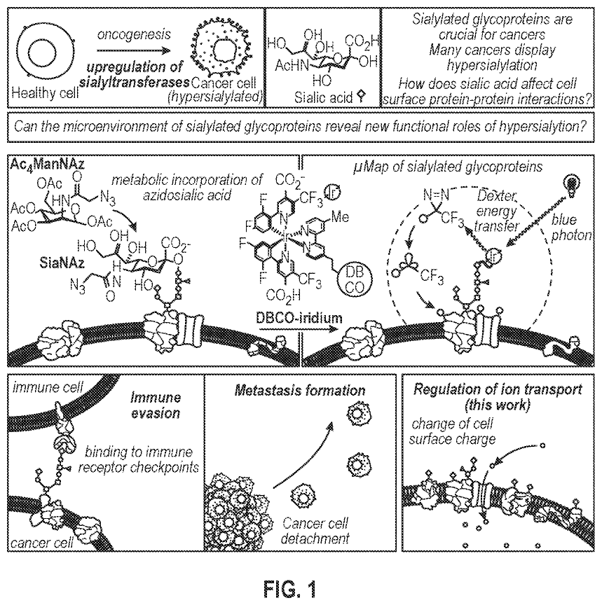

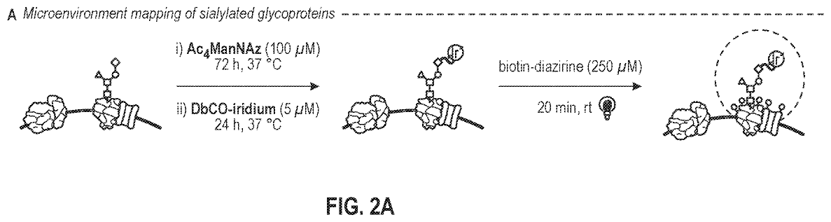

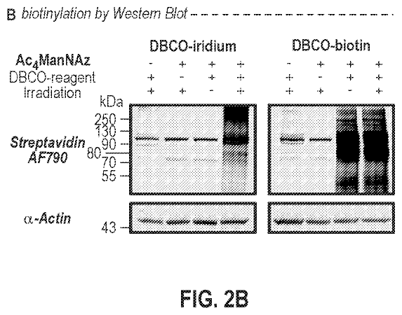

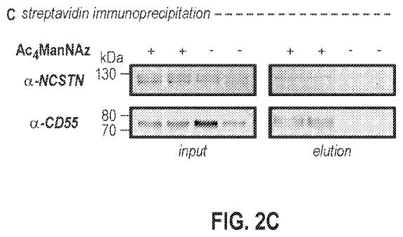

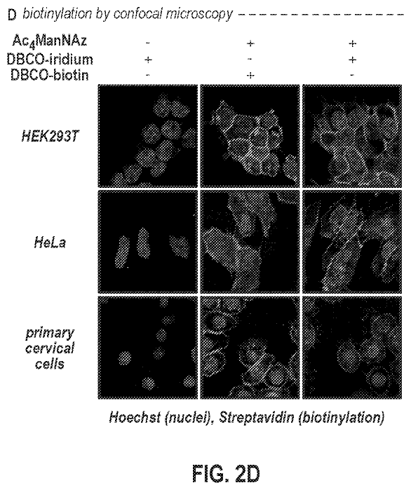

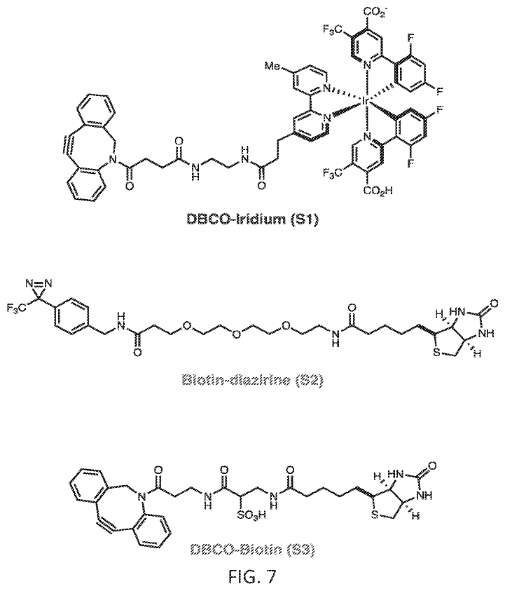

An iridium photocatalyst was developed for microenvironment mapping to unravel the interactome of sialylated cell-surface glycoproteins, as illustrated in FIG. 1. The study employing the iridium photocatalyst was initiated according to FIG. 2A. Incubation of HeLa cells with tetraacetyl-N-azidoacetylmannosamine (Ac4ManNAz), followed by treatment with DBCO-iridium (FIGS. 7, S1) led to incorporation of the iridium photocatalyst onto glycoproteins. Irradiation in the presence of biotin-diazirine (FIGS. 7, S2) resulted in cell-surface biotinylation as observed by western blot. (FIG. 2B). Control reactions displayed minimal biotinylation when omitting the azidosugar, DBCO-iridium reagent, or blue light irradiation. Importantly, immunoprecipitation over streptavidin beads showed strong enrichment of known sialylated glycoproteins nicastrin (NCSTN) and complement decay-accelerating factor (CD55), providing confidence in our workflow (FIG. 2C).

This method was compared to direct biotinylation of Ac4ManNAz using DBCO-biotin (S3) and observed labeling of the sialome via western blot. These results suggest our catalytic labeling method can install ˜0.7 tags per catalyst (see ESI), representing a significant improvement over existing PAL probes, which typically react with water (>95%) and display minimal protein labeling. Lastly, this protocol was also applicable to both HEK293T cells and primary cervical cells (PCC) (FIG. 2D), highlighting the versatility of the workflow. In all cases, confocal microscopy revealed strong biotinylation after treatment with either DBCO-iridium or DBCO-biotin (FIGS. 7, S3) and irradiation in presence of biotin-diazirine. In the absence of azido-sugar no labeling was observed in any case.

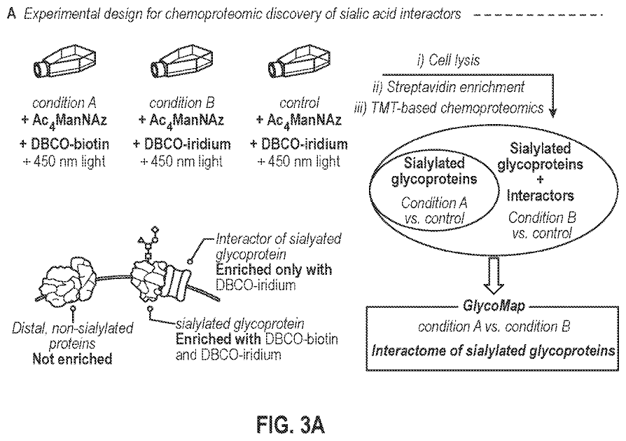

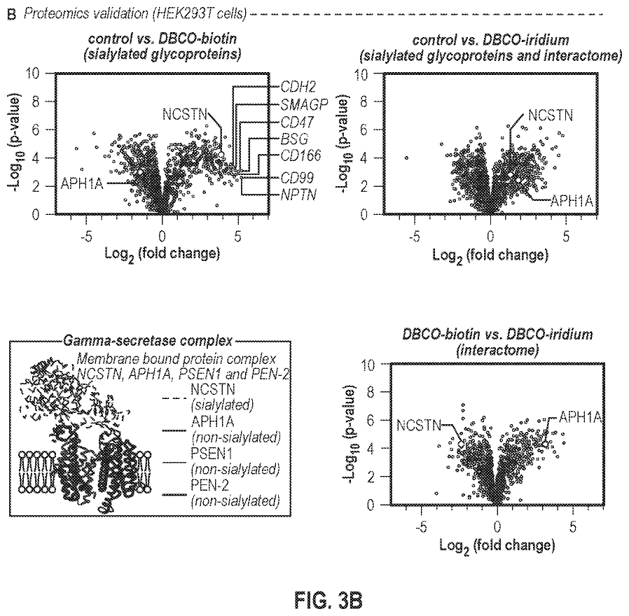

Encouraged by these results, a tandem mass tag (TMT)-based quantitative chemoproteomics workflow was developed to identify sialylated cell-surface glycoproteins and map their interactomes (FIG. 3A). For each cell type we performed comparative experiments using three different conditions: the first condition (condition A) included SPAAC with DBCO-biotin, which results in biotinylation of only sialylated glycoproteins. Condition B utilized SPAAC with DBCO-iridium for biotinylation of sialylated glycoproteins and their cognate interactome via uMap. Lastly, a control experiment was performed with DBCO-iridium in the absence of Ac4ManNAz. Together, these parameters allow for the identification of 1) the sialylated glycoproteins, 2) sialylated glycoproteins and their local interactome, and 3) selective identification of protein interactors of the cell surface sialome (GlycoMap). We first examined this chemoproteomic workflow on HEK293T cells (FIG. 3B). Using condition A (vs. control) we found significant enrichment (>1.5 Log₂(fold change); >1.5−Log₁₀(p-value)) of 363 proteins, of which 93% were known glycoproteins, including nicastrin (NCSTN), cadherin 2 (CDH2), small cell adhesion glycoprotein (SMAGP), cluster of differentiation 47 (CD47), basignin (BSG), cluster of differentiation 166 (CD166), cluster of differentiation 99 (CD99) and neuroplastin (NPTN). As predicted by FIG. 3A, condition B (vs. control) enriches both sialylated glycoproteins as well as proximal proteins and thus shares an ˜65% overlap with the proteins enriched in condition A (vs. control) (FIG. S9). Analysis of the sialic acid interactome generated by GlycoMap showed 81% of the enriched proteins were membrane-associated, reflecting the accuracy of our labeling approach. In addition, several lysosomal proteins (21) were enriched, presumably arising via internalization of iridium-bound glycoproteins prior to proximity labeling.

In the initial analysis of the GlycoMap dataset a known membrane-bound protein complex, gamma secretase, was examined in order to validate our methodology. This heterotetramer of membrane proteins (NCSTN, APHIA, PSENI, PEN-2) proteolytically cleaves many integral membrane proteins, but only NCSTN is directly sialylated. In our dataset, NCSTN is highly enriched (3.8 log₂ FC) in condition A (vs. control), whereas the non-sialylated interactor, APHIA, is strongly enriched (3.3 log₂ FC) in the GlycoMap arm demonstrsating the pMap workflow can delineate between sialylated glycoproteins and their interactors.

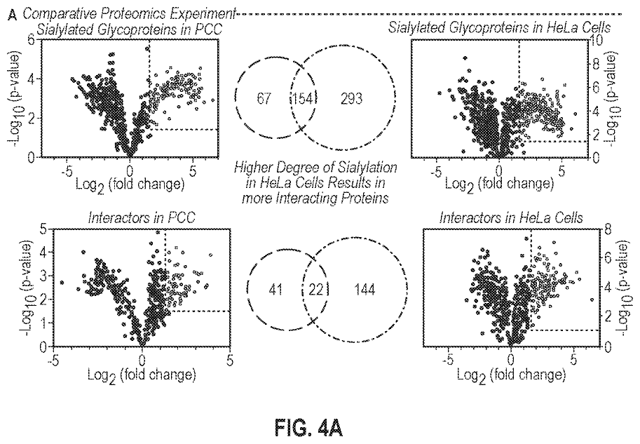

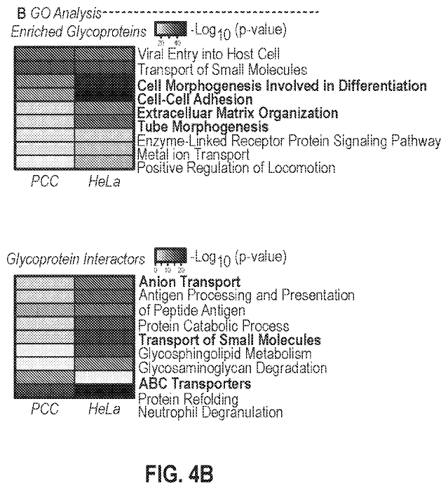

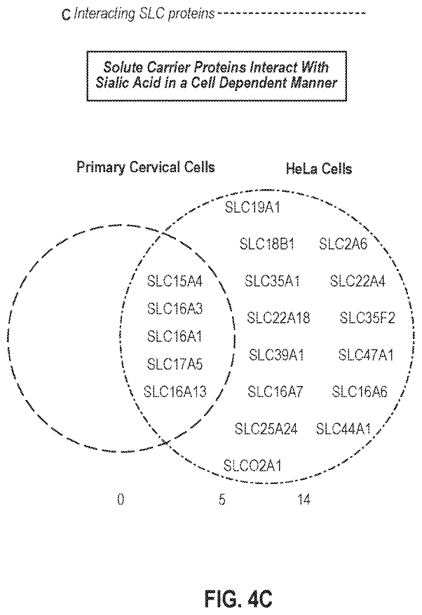

With an established workflow in hand, it was next sought to investigate hypersialylation events in oncogenesis. To examine this, we performed comparative GlycoMap experiments on both primary cervical cancer cells (PCC) and the HeLa cervical adenocarcinoma cell line (FIG. 4A).

In agreement with previous observations of upregulated sialylation, chemoproteomic analysis revealed significantly higher sialylation in HeLa cells (447 enriched proteins) than in PCC (223 enriched proteins) (FIG. 4A). This sialome increase in HeLa cells consequently yielded a higher number of interacting proteins (166 enriched proteins in HeLa cells vs 63 enriched proteins in PCC).

Global gene ontology (GO) analysis was then performed to categorize the enriched sialylated proteins and their interactors (FIG. 4B). Functional enrichment from both cell types was in good agreement with the known roles of identified glycoproteins: cell adhesion, host-cell entry, and regulation of migration, death and defense. Furthermore, examination of GO terms that differed significantly (≥10 Log₁₀ (p-value) change) between the primary and cancerous cervical cells validated that the sialylated glycoproteins in cancerous cells are related to typical oncological phenotypes, including cell morphogenesis, cell-cell adhesion, extracellular matrix organization and tube morphogenesis.

Interestingly, when comparing the roles of identified sialic acid interacting proteins, terms related to small molecule transport were clearly enriched in HeLa cells (organic ion transport, transport of small molecules, vitamin transport), and we specifically noted numerous enriched solute carrier proteins (SLCs) in this dataset (FIG. 4C). In particular, interactions of sialylated proteins with SLCs associated with ethanolamine, carnitine, and zinc transport were all significantly enriched in HeLa cells over PCC.

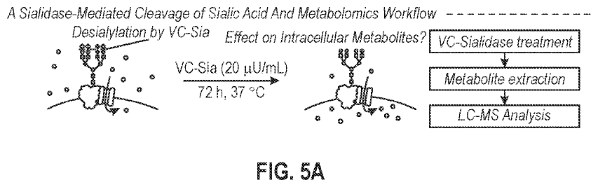

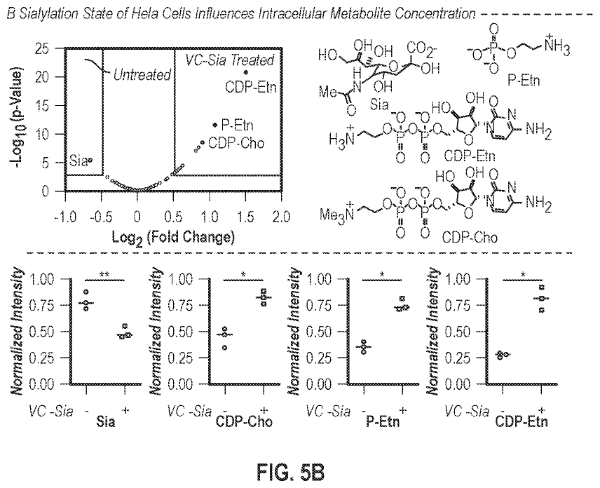

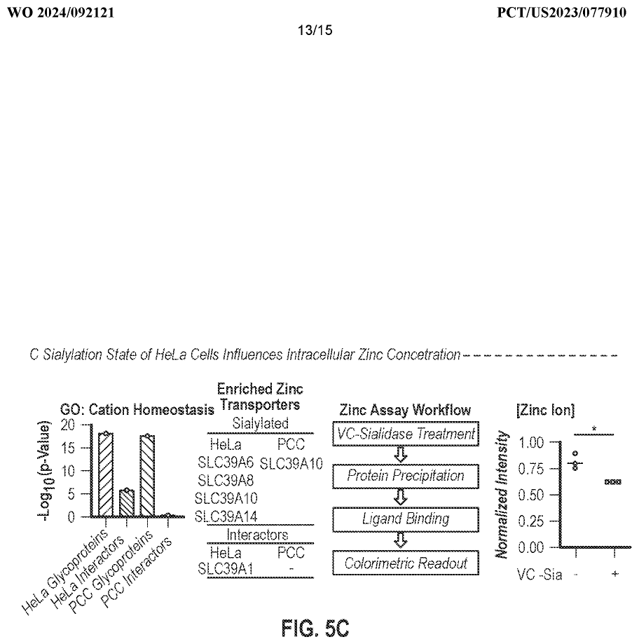

To explore potential consequences of these interactions, we looked for the metabolic consequences of enzymatically depleting them (FIG. 5A). Treatment of HeLa cells with sialidase isolated from Vibrio cholerae (VC-Sia) efficiently cleaves sialic acids α2,3-, α2,6-, or α2,8-linked to cell surface glycans, enabling us to modulate global sialylation status. We incubated HeLa cells either in presence or absence of VC-Sia and then performed mass spectrometry-based metabolomic quantification on cellular metabolite extracts. While most metabolite levels were minimally affected by sialidase treatment, we found that levels of ethanolamine derivatives, including cytidine diphosphate ethanolamine (CDP-Etn) phosphate ethanolamine (P-Etn) and cytidine diphosphate choline (CDP-choline) were significantly increased in sialidase-treated cells (FIG. 5B). The solute carrier protein responsible for the transport of ethanolamine, choline-like transporter 1 (SLC44A1), is not known to be glycosylated, however our dataset suggests that its function could be regulated by neighboring sialylated glycoproteins. Based on these results, we hypothesize that cell-surface sialic acids may present a negatively charged surface around membrane bound transporters, which in turn could affect the transport of ions, including metabolites.

Similarly, we also investigated the impact of sialylation on zinc uptake (FIG. 5C). Zinc is imported through the cell membrane by a series of solute carrier proteins of the SLC39 family, four of which are shown in our HeLa dataset to be sialylated (SLC39A6, SLC39A8, SLC39A10 and SLC39A14) and one (SLC39A1) is suggested to interact with a sialylated glycoprotein. Zinc is a key micronutrient that plays a significant role in cell function and its transport is dysregulated in many cancers.

Using a colorimetric assay to determine the zinc level of untreated and sialidase-treated HeLa cells, we found that the zinc level was significantly higher in cells treated with VC-Sia. These data suggest that cell surface sialylation and/or the interaction with sialylated glycoproteins, plays a role in the regulation of cellular zinc concentration.

In conclusion, hypersialylation in cancer has recently attracted significant interest from the academic and pharmaceutical sectors. However, tools to understand the biochemical consequences of hypersialylation remain limited. Here, we have described a novel proximity labeling platform to identify sialylated cell-surface glycoproteins and their interactors. This sensitive and precise method is robust and compatible with various cell lines, including primary cells. Our comparative proteomics studies between primary and cancerous cervical cell lines show a significant link between sialylation and solute carrier proteins, suggesting a new role for sialylation. Metabolomics data suggest that these interactions regulate the function of certain solute carriers. Together, our platform represents a powerful new approach to sialic acid interactome profiling, providing a systems-level tool for the elucidation of hypersialylation's biochemical consequences.

### Materials

All buffers and materials were used as received from commercial sources. Tetraacetyl N-Azidoacetylmannosamine (Ac4ManNAz) (900917), Bovine serum albumin (BSA) (A7906), and Eppendorf Protein LoBind tubes (Z666505) were purchased from Millipore Sigma (St. Louis, MO). DBCO-Sulfo-Link-biotin (DBCO-biotin) (BP-22296) was purchased from Broadpharm (San Diego, CA). Biotin-(peg)₃-diazirine (biotin-diazirine) and [Ir(dCO₂HdFCF₃ppy)₂(bpy-dbco) (DBCO-iridium) were synthesized as described previously^{1,2} RIPA Buffer (89900), 1×DPBS (14190144), Pierce BCA Protein Assay Kit (23227), and iBright Prestained Protein ladder (LC5615) were purchased from Thermo Scientific (Rockford, IL). TBST (IBB-581X) was purchased from Boston BioProducts (Ashland, MA). 12% Criterion TGX precast gels (5671044) and 4× Laemmli sample buffer (161-0747) were purchased from BioRad (Hercules, CA). Poly-L-Lysine Solution was obtained from Sigma-Aldrich (St. Louis, MO). Paraformaldehyde (16% solution) was obtained from Thermo Fisher Scientific (Rockford, IL). Streptavidin-Alexa Fluor 488 was obtained from BioLegend (San Diego, CA). Standard Tissue Culture Dishes were obtained from Thermo Fisher Scientific (Waltham, MA). DPBS (Gibco, #14190250), DMEM high glucose (Gibco, #31053036), DMEM high glucose—no phenol red (Gibco, #31053028), Fetal Bovine Serum (Gibco, #10437-028), Penicillin-Streptomycin (Gibco, #15070063), Trypsin-EDTA (Gibco, #25300054), Trypsin protease MS (Pierce, #PI90057), and RIPA buffer (Thermo, #89900) were obtained from Thermo Fisher Scientific. PMSF (Sigma Aldrich, #78830) and complete EDTA free protease inhibitor (Roche, #11873580001) were obtained from Sigma Aldrich. Streptavidin Magnetic Beads were obtained from Thermo Fisher Scientific (Pierce, #88816). Trifluoroacetic acid (Optima grade), Acetonitrile (Optima grade), Water (Optima grade), and Acetic acid (Optima grade) were obtained from Thermo Fisher Scientific.

Triethylammonium bicarbonate (1M Sigma Aldrich, #90360), 50% Hydroxylamine solution (Sigma Aldrich, #438227), Ammonium hydrogen carbonate (LiChropur, Merck, #5438350), and Iodoacetamide (Sigma Aldrich, #11149) were obtained from Sigma Aldrich. TMT10plex kits (Thermo), Urea (Pierce, Sequanal, #29700), and DTT (Thermo, #R0862) were obtained from Thermo Fischer Scientific.

### Cell Lines

HEK293 (CRL321) and HeLa cells (CCL2) were obtained from American Type Culture Collection (ATCC) and were cultured in Dulbecco's Modified Eagle Medium (DMEM) high glucose (Gibco, #31053036) supplemented with 10% Fetal Bovine Serum (Gibco, #10437-028) and 1% Penicillin Streptomycin (Gibco, #15070063) at 37° C. and 5% CO₂ atmosphere in 10 cm dishes.

Primary cervical epithelial cells (ATCC, #PCS-480-011) were cultured in the recommended medium (ATCC, #PCS-480-032), supplemented with the recommended growth kit (ATCC, #PCS-480-042) according to the protocol provided by ATCC.

### Antibodies

anti-NCSTN (Rabbit, polyclonal): Invitrogen (#PA5-17735)
anti-CD55 (Rabbit, polyclonal): Invitrogen (#PA5-29657)
anti-Actin (Mouse, monoclonal): Cell Signaling Technologies (#37005)

### Glycomap Experiments

The general experimental workflow is illustrated in FIG. 2A.

### Optimization of the Glycomap Conditions

HEK293T cells (approx. 0.4×10⁶ cells) were incubated in 6 well dishes in complete DMEM (2 mL) in presence or absence of Ac4ManNAz (100 μM) at 37° C. for 72 hours. The cells were washed with DPBS (3×1 mL) and then incubated in complete DMEM (2 mL) containing DBCO-iridium (2.5-10.0 μM) at 37° C. for 3-24 hours. The cells were washed with DPBS (3×1 mL) and irradiated in DMEM (no phenol-red, 200 μL) containing biotin-diazirine (250 μM) at room temperature for 20 minutes in the biophotoreactor (blue LEDs). The cells were washed with DPBS (3×1 mL), scraped in DPBS (1 mL), and transferred to 1.5 mL Eppendorf tubes. The cells were pelleted at 400×G for 5 minutes and resuspended in Ripa lysis buffer (500 μL). The cells were lysed by sonication (bioruptor) at 4° C. for 10 minutes (20 cycles, 15 sec on 15 sec off at 100% power). The protein concentration was normalized by BCA assay and the lysate was analyzed by Western blot (10 μg of protein per lane, 12% gel, 150 V). The gel was transferred via iBlot 2 to an NC membrane. Following transfer, the membrane was stained with total protein stain, washed with washing solution (3×5 sec) and imaged via Li-Cor Odyssey CLx scanner in the 700 nm channel. The membrane was then immersed in Odyssey Blocking Buffer (Li-Cor, 927-50000) and incubated at room temperature for 1 hour. The blocking solution was decanted, and 10 mL of fresh blocking buffer containing 0.5 μL of IRDye 800CW streptavidin (Li-Cor, 926-32230) was added. This mixture was rocked for 60 minutes. The buffer was decanted, and the membranes were washed with 1×TBST (4×5 min) and water (3×5 sec) before imaging via Li-Cor Odyssey CLx scanner in the 800 nm channel. The labeling efficiency was assessed by densitometry by comparing against an experiment which was performed without any Ac4ManNAz.

### Western Blot Analysis

HeLa cells (approx. 0.4×10⁶ cells) were incubated in 6 well dishes in complete DMEM (2 mL) containing Ac4ManNAz (100 μM) at 37° C. for 72 hours. The cells were washed with DPBS (3×1 mL) and then incubated in complete DMEM (2 mL) containing DBCO-iridium (5 μM) or DBCO-biotin (5 μM) at 37° C. for 24 hours. The cells were washed with DPBS (3×1 mL) and irradiated in DMEM (no phenol-red, 200 μL) containing biotin-diazirine (250 μM) at room temperature for 20 minutes in the biophotoreactor (blue LEDs). The cells were washed with DPBS (3×1 mL), scraped in DPBS (1 mL), and transferred to 1.5 mL Eppendorf tubes. The cells were pelleted at 400×G for 5 minutes and resuspended in Ripa lysis buffer (500 μL). The cells were lysed by sonication (bioruptor) at 4° C. for 10 minutes (20 cycles, 15 sec on 15 sec off at 100% power). The protein concentration was normalized by BCA assay and the lysate was analyzed by Western blot (20 μg of protein per lane, 12% gel, 150 V). The gel was transferred via iBlot 2 to an NC membrane Following transfer, the membrane was immersed in Odyssey Blocking Buffer (Li-Cor, 927-50000) and incubated at room temperature for 1 hour. The blocking buffer was replaced with fresh blocking buffer (10 mL) containing anti-Actin antibody (10 μL) and the membrane was rocked for 1 hour. The buffer was decanted, and the membranes were washed with 1×TBST (4×5 min) and water (3×5 sec). The blocking solution was decanted, and 10 mL of fresh blocking buffer containing 1 μL of IRDye 680RD Goat anti-Mouse IgG secondary antibody (Li-Cor, 926-68070), and 1 μL of IRDye 800CW streptavidin (Li-Cor, 926-32230) was added. This mixture was rocked for 60 minutes. The buffer was decanted, and the membranes were washed with 1×TBST (4×5 min) and water (3×5 sec) before imaging via Li-Cor Odyssey CLx scanner in the 700 nm and 800 nm channel.

### Confocal Microscopy

Cells (HEK293T, HeLa or primary cervical epithelial cells) (approx. 2×10⁴ cells) were incubated in poly-lysine coated 8 well chamber slides in complete DMEM (200 μL) containing Ac4ManNAz (100 μM) at 37° C. for 48 hours. The cells were washed with DPBS (3×200 μL) and then incubated in complete DMEM (200 μL) containing DBCO-iridium (5 μM) or DBCO-biotin (5 μM) at 37° C. for 24 hours. The cells were washed with DPBS (3×200 μL) and irradiated in DMEM (no phenol-red, 200 μL) containing biotin-diazirine (250 μM) at room temperature for 20 minutes in the biophotoreactor (blue LEDs). The cells were washed with DPBS (3×200 μL) and fixed with prewarmed 4% paraformaldehyde (200 μL) at room temperature for 30 min. The cells were washed with DPBS (2×200 μL) and blocked with 3% BSA in DPBS (200 μL) at room temperature for 1 hour. The blocking buffer was replaced with fresh 3% BSA in DPBS (200 μL) containing Hoechst (1:1000), and Streptavidin-AlexaFluor 555 conjugate (1:1000). The cells were stained in the dark at room temperature for 1 hour and then stored in the dark at 4° C. The cells were imaged on a NIKON A1R-SI microscope (Nikon Instruments, Inc., Melville, NY) at 20× magnification. The images were processed with Fiji-ImageJ. Images shown are representative of the multiple cross-sectional images taken during each session.

### Streptavidin Immunoprecipitation

HeLa cells (approx. 5×10⁶ cells) were incubated in 10 cm dishes in complete DMEM (10 mL) containing Ac4ManNAz (100 μM) at 37° C. for 72 hours. The cells were washed with DPBS (3×5 mL) and then incubated in complete DMEM (5 mL) containing DBCO-iridium (5 μM) at 37° C. for 24 hours. The cells were washed with DPBS (3×5 mL) and irradiated in DMEM (no phenol-red, 5 mL) containing biotin-diazirine (250 μM) at room temperature for 20 minutes in the biophotoreactor (blue LEDs). The cells were washed with DPBS (3×5 mL), scraped in DPBS (5 mL), and transferred to 15 mL conical tubes. The cells were pelleted at 400×G for 5 minutes and resuspended in Ripa lysis buffer (1 mL) containing protease inhibitor cocktail. The cells were lysed by sonication (bioruptor) at 4° C. for 10 minutes (20 cycles, 15 sec on 15 sec off at 100% power).

The protein concentration was normalized by BCA assay. To Pierce Streptavidin beads (80 μL) was added lysate (0.75 mg protein/experiment) and the beads were inverted at 4° C. for 16 hours. The beads were washed with 1% SDS (3×500 μL, 5 minutes per wash), 1M NaCl (3×500 μL) and 10% EtOH (3×500 μL). The proteins were then eluted with elution buffer/laemmli (3:1, 40 μL) and boiled at 95° C. for 15 minutes. The mother liquor was separated hot and analyzed by western blot and compared against lysate input (10 μg of protein per input lane) (12%, 150 V).

The gel was transferred via iBlot 2 to an NC membrane Following transfer, the membrane was immersed in Odyssey Blocking Buffer (Li-Cor, 927-50000) and incubated at room temperature for 1 hour. The blocking buffer was replaced with fresh blocking buffer (10 mL) containing anti-CD55 antibody (10 μL) and the membrane was rocked at 4° C. for 16 hours. The buffer was decanted, and the membranes were washed with 1×TBST (4×5 min) and water (3×5 sec). The blocking solution was decanted, and 10 mL of fresh blocking buffer containing 1 μL of IRDye 800CW Goat anti-Rabbit IgG Secondary Antibody (Li-Cor, 926-32211) was added. This mixture was rocked at room temperature for 60 minutes. The buffer was decanted, and the membrane was washed with 1×TBST (4×5 min) and water (3×5 sec) before imaging via Li-Cor Odyssey CLx scanner in the 800 nm channel.

For subsequent staining, the membrane was stripped using Restore PLUS Western Blot Stripping Buffer (Thermo Fisher Scientific, 46430) at room temperature for 30 minutes. The membrane was blocked and stained as described above using anti-NCSTN antibody (1:1000).

### Proteomics Workflow

HeLa cells (approx. 5×10⁶ cells) were incubated in 10 cm dishes in complete DMEM (10 mL) containing Ac4ManNAz (100 μM) at 37° C. for 72 hours. The cells were washed with DPBS (3×5 mL) and then incubated in complete DMEM (5 mL) containing DBCO-iridium (5 μM) at 37° C. for 24 hours. The cells were washed with DPBS (3×5 mL) and irradiated in DMEM (no phenol-red, 5 mL) containing biotin-diazirine (250 μM) at room temperature for 20 minutes in the biophotoreactor (blue LEDs). The cells were washed with DPBS (3×5 mL), scraped in DPBS (5 mL), and transferred to 15 mL conical tubes. The cells were pelleted at 400×G for 5 minutes and resuspended in Ripa lysis buffer (1 mL) containing protease inhibitor cocktail. The cells were lysed by sonication (bioruptor) at 4° C. for 10 minutes (20 cycles, 15 sec on 15 sec off at 100% power).

The protein concentration was normalized by BCA assay. To Pierce Streptavidin beads (200 μL) was added lysate (2.0 mg protein/experiment) and the beads were inverted at 4° C. for 16 hours. The beads were washed with 1% SDS (3×500 μL, 5 minutes per wash), 1M NaCl (3×500 μL) and 10% EtOH (3×500 μL). The beads were resuspended in RIPA buffer (500 μL) and transferred to a new 1.5 mL Lo-bind tube.

The supernatant was removed, and the beads were washed with DPBS (3×500 μL) and NH₄HCO₃ (100 mM) (3×500 μL). The beads were resuspended in 6 M urea in DPBS (500 μL) and 200 mM DTT in 25 mM NH₄HCO₃ (25 μL) was added. The beads were inverted at 55° C. for 30 minutes. Subsequently, 500 mM iodoacetamide in 25 mM NH₄HCO₃ (30 μL) was added and the beads were inverted for 30 minutes at room temperature in the dark. The supernatant was removed, and the beads were washed with DPBS (3×500 μL) and TEAB (50 mM) (3×500 μL). The beads were resuspended in TEAB (500 μL) and transferred to a new protein LoBind tube, pelleted, and the supernatant removed.

The beads were resuspended in 50 mM TEAB (40 μL) and trypsin (1 mg/mL in 50 mM acetic acid; 1.2 μL) was added and the beads were inverted overnight at 37° C. After 16 hours, additional trypsin (0.8 μL) was added, and the beads were inverted for an additional 1 hour at 37° C. The beads were subsequently pelleted. Meanwhile, the TMT10 plex label reagents (0.8 mg) (Thermo) were equilibrated to room temperature, diluted with anhydrous acetonitrile (Optima grade; 41 μL, 5 minutes with vortexing), and centrifuged to gather the contents.

Then each set of trypsinized peptides was added to the corresponding TMT label (40 μL in TEAB added to 41 μL in MeCN). The beads were then washed with further TEAB (20 μL) to collect remaining peptides. The labeling reaction was allowed to proceed for 2 hours at room temp. The samples were then quenched with 5% hydroxylamine (8 μL) and incubated at room temperature for 15 minutes. The samples were pooled in a new Protein LoBind tube and quenched with TFA (16 μL, Optima). The samples were stored at −80° C. until proteomics were conducted. Samples were desalted and fractionated prior to running.

### Labeling Efficiency

Based on the Western blot results shown in FIG. 2B the efficiency of labeling via Glycomap can be calculated using densitometry. In this calculation it is assumed that the SPAAC efficiency of DBCO-iridium is comparable to that of DBCO-biotin. Using densitometry on the western blot stained with streptavidin (MW range from 45 kDa to top of blot) we calculate approximately 1 tag per catalyst.

The whole calculation is listed below:
Densitometry readout DBCO-Iridium full experiment: 108′442
Densitometry readout DBCO-Iridium control: 6′164
Experiment/control: 17.6
Densitometry readout DBCO-Biotin full experiment: 158′335
Densitometry readout DBCO-Biotin control: 5′937
Experiment/control: 26.7
Comparison: 17.6/26.7=0.7 tags per catalyst.

### Metabolomics Experiments

### Metabolite Extraction

In transparent 10 cm dishes (×6), HeLa cells were grown for 72 hours in 5 mL complete DMEM supplemented with 10% FBS in presence or absence of neuraminidase (20 U/mL). After 72 hours, the cells were washed with DPBS (3×5 mL) and incubated with TrypLE (1 mL) for 15 minutes at 37° C. The cells were transferred to 15 mL conical tubes using 4 mL DPBS to wash the plates. The cells were pelleted at 500×G for 4 minutes and resuspended in 1 mL DPBS. The cells were counted, and 500′000 cells/experiment were transferred to 1.5 mL Eppendorf tubes. The cells were pelleted (500×g for 4 min), the supernatant removed, and the cells were lysed with ice cold 80% MeOH (60 μL) for 30 minutes at 0° C. The cell lysate was clarified by centrifugation at 20′000×G for 25 minutes and the lysates were transferred to new 0.5 mL Eppendorf tubes and stored at −80° C. until mass spectrometry analysis.

### LC-MS

HPLC-grade water, methanol, and acetonitrile were obtained from Thermo Fisher Scientific. Supernatant sample was thawed at room temperature and kept at 4° C. in an autosampler. Samples were analyzed using a Q Exactive Plus mass spectrometer coupled to Vanquish UHPLC system (Thermo Fisher Scientific). LC separation was achieved using a XBridge BEH Amide column (2.1 mm×150 mm, 2.5-μm particle size, 130-A pore size; Waters, Milford, MA, USA) using a gradient of solvent A (20 mM ammonium acetate+20 mM ammonium hydroxide in 95:5 water/acetonitrile [pH 9.45]) and solvent B (acetonitrile). Flow rate was 150 l/min. The gradient was 0 min, 90% B; 2 min, 90% B; 3 min, 75%; 7 min, 75% B; 8 min, 70%, 9 min, 70% B; 10 min, 50% B; 12 min, 50% B; 13 min, 25% B; 14 min, 25% B; 16 min, 0% B, 20.5 min, 0% B; 21 min, 90% B; 25 min, 90% B. The column temperature was 25° C., and the injection volume was 5 μL. The mass spectrometer is operated in full scan mode with separate runs in positive mode and negative mode covering m/z 70-1000, with resolution 140,000 at m/z 200, AGC target 5E6, maximum injection time 200 ms.

### Data Analysis

Data analyses were performed using El-Maven and the metabolites identified with an in-house library using authentic standards.³ Metabolite signal intensity were then further processed in Excel (median correction) and Graphpad Prism (transformation, t-test, and volcano plot generation).

### Colorimetric Zinc Assay

The zinc assay was purchased from Abcam (ab102507), and the experiment was performed according to the procedure below, adapted from the supplier's instructions.

In transparent 10 cm dishes, HeLa cells were grown in complete medium (DMEM, supplemented with 10% FBS) in presence (×3) or absence (×3) of VC-sialidase (20 mU/mL medium) for 96 hours. The medium was exchanged every 24 hours and fresh sialidase was added at those timepoints. After 96 hours, the medium was removed and DPBS (5 mL) was added. The cells were scraped, transferred to 15 mL conical tubes, and pelleted at 400×g for 5 minutes. The supernatant was removed, and the cells were lysed in 50 μL EDTA-free lysis buffer lysed by sonication (bioruptor) at 4° C. for 10 minutes.

The lysate was clarified at 18′000×g for 15 minutes and analyzed by BCA assay. 30 L of the cell lysate was transferred to a 1.5 mL Eppendorf tube containing 30 μL of 7% TCA to precipitate proteins. The mixture was clarified at 18′000×g for 5 minutes and 50 L of the resulting solution were used for the zinc detection assay.

### Zinc Concentrations (Nmol Zinc Mg of Protein-Free Lysate):

1 (untreated): 1.7 nmol/mg
2 (untreated): 2.0 nmol/mg
3 (untreated): 1.8 nmol/mg
4 (Sia-treated): 1.4 nmol/mg
5 (Sia-treated): 1.4 nmol/mg
6 (Sia-treated): 1.4 nmol/mg

### Comparison of Zinc Levels in PCC and HeLa Cells

In transparent 10 cm dishes, HeLa cells (×3) and PCC cells (×3) were grown in complete medium for primary cervical cells (ATCC, PCS-480-032) for 24 hours. The medium was removed and DPBS (5 mL) was added. The cells were scraped, transferred to 15 mL conical tubes, and pelleted at 400×g for 5 minutes. The supernatant was removed, and the cells were lysed in 50 L EDTA-free lysis buffer lysed by sonication (bioruptor) at 4° C. for 10 minutes.

The lysate was clarified at 18′000×g for 15 minutes and analyzed by BCA assay. 30 μL of the cell lysate was transferred to a 1.5 mL Eppendorf tube containing 30 μL of 7% TCA to precipitate proteins. The mixture was clarified at 18′000×g for 5 minutes and 50 L of the resulting solution were used for the zinc detection assay.

### Zinc Concentrations (Nmol Zinc/Mg of Protein-Free Lysate):

1 (PCC): 2.7 nmol/mg
2 (PCC): 2.7 nmol/mg
3 (PCC): 3.0 nmol/mg
4 (HeLa): 2.3 nmol/mg
5 (HeLa): 2.5 nmol/mg
6 (HeLa): 2.3 nmol/mg

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A conjugate comprising:
a transition metal catalyst coupled to a cell surface glycoprotein.

2. The conjugate of claim 1, wherein the transition metal catalyst is coupled to the glycoprotein via a derivatized sialic acid linker.

3. The conjugate of claim 2, wherein the transition metal catalyst and derivatized sialic acid linker are coupled via click chemistry.

4. The conjugate of claim 1, wherein the transition metal complex is of the formula: 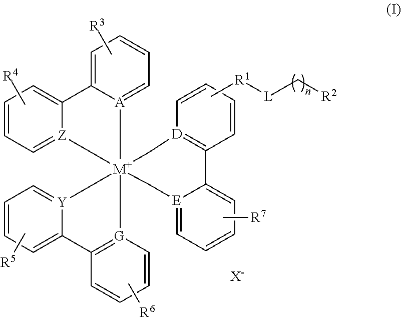
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

5. The conjugate of claim 4, wherein R² is a click chemistry moiety selected from the group consisting of DBCO, BCN, TCO, tetrazine, alkyne and azide.

6. The conjugate of claim 1, wherein the M is a platinum group metal.

7. The conjugate of claim 6, wherein the M is iridium.

8. The conjugate of claim 1, wherein the transition metal catalyst is a photocatalyst.

9. The conjugate of claim 1, wherein the transition metal catalyst has electronic structure for energy transfer to a protein labeling agent.

10. The conjugate of claim 9, wherein the energy transfer is Dexter energy transfer.

11. A system for profiling microenvironments local to the sialylated proteome comprising:
a protein labeling agent; and
a conjugate comprising a transition metal catalyst coupled to a cell surface glycoprotein via a derivatized sialic acid linker, wherein the transition metal catalyst has electronic structure permitting energy transfer to the protein labeling agent to provide a reactive intermediate.

12. The system of claim 11, wherein the reactive intermediate is operable to label a protein within a predetermined radius of the conjugate.

13. The system of claim 11, wherein the transition metal catalyst and derivatized sialic acid linker are coupled via click chemistry.

14. The system of claim 11, wherein the transition metal complex is of the formula: 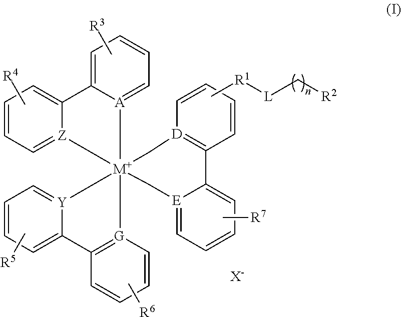
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is an optional linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

15. The system of claim 14, wherein R² is a click chemistry moiety selected from the group consisting of DBCO, BCN, TCO, tetrazine, alkyne and azide.

16. The system of claim 14, wherein the M is a platinum group metal.

17. The system of claim 16, wherein the M is iridium.

18. The system of claim 11, wherein the transition metal catalyst is a photocatalyst.

19. The system of claim 11, wherein the energy transfer is Dexter energy transfer.

20. The system of claim 11, wherein the protein labeling agent is a diazirine.

21. The system of claim 20, wherein the diazirine comprises a molecular marker.

22. A method of profiling microenvironments local to the sialylated proteome comprising:
forming a conjugate comprising a transition metal catalyst coupled to a cell surface glycoprotein via a derivatized sialic acid linker;
activating a protein labeling agent to a reactive intermediate with the transition metal catalyst; and
coupling the reactive intermediate to a protein within a predetermined radius of the conjugate.

23. The method of claim 22, wherein activating the protein labeling agent to the reactive intermediate comprises energy transfer from the transition metal catalyst to the protein labeling agent.

24. The method of claim 22, wherein the protein labeling agent is a diazirine.

25. (canceled)

26. The method of claim 22, wherein the predetermined radius is 2-100 nm.

27-33. (canceled)
